(19) United States
(12) Patent Application Publication
(10) Pub. No.: US 2024/0090871 A1
(43) Pub. Date: Mar. 21, 2024

(54) Ultrasound Transmissive Article

(71) Applicant: Rivanna Medical, Inc., Charlottesville, VA (US)

(72) Inventors: Adam Dixon, Charlottesville, VA (US); Zachary Leonard, Charlottesville, VA (US); Paul Sheeran, Charlottesville, VA (US); Frank William Mauldin, JR., Troy, VA (US)

(21) Appl. No.: 18/371,027

(22) Filed: Sep. 21, 2023

Related U.S. Application Data
(60) Provisional application No. 63/408,490, filed on Sep. 21, 2022.

(51) Int. Cl. A61B 8/00

(52) U.S. Cl. CPC A61B 8/4281

(57) ABSTRACT

An ultrasound transmission material that may be used to convey ultrasonic energy in medical ultrasound or non-destructive testing (NDT) applications.

### CROSS-REFERENCE TO RELATED APPLICATION

The present application relies on the disclosures of and claims priority to and the benefit of the filing date of U.S. Application No. 63/408,490, filed Sep. 21, 2022.

The disclosures of the above application are hereby incorporated by reference herein in their entireties.

### STATEMENT REGARDING FEDERALLY SPONSORED RESEARCH

This invention was made with government support under Contract No. 75A50121C00035 awarded by the Department of Health and Human Services; Office of the Assistant Secretary for Preparedness and Response; Biomedical Advanced Research and Development Authority. The government has certain rights in the invention.

### TECHNICAL FIELD

The present invention is related to an ultrasound transmission material that may be used to convey ultrasonic energy in medical ultrasound or non-destructive testing (NDT) applications.

### BACKGROUND

The use of ultrasonic energy for imaging in medical and non-destructive testing applications is well established. Distance measurements may be acquired by emitting ultrasonic energy into a patient or test material and measuring the time-of-flight of the reflected ultrasonic echoes. Most often, an ultrasound transducer is placed in direct contact with the imaging target, and a thin layer of liquid or gel is used to convey the ultrasonic energy from the transducer into the imaging target. However, liquid and gel coupling materials are often incapable of providing suitable contact between the ultrasound transducer and imaging targets with irregularly shaped surfaces. In these situations, a thicker, conformable ultrasound transmission material is frequently placed in between the ultrasound transducer and the imaging target to provide suitable contact for the transmission of ultrasonic energy.

Conformable ultrasound transmission materials are most often comprised of thermoplastic or thermoset elastomeric materials, including hydrogels, silicones, polyurethanes, and olefins. These elastomeric materials frequently include additives, fillers, and plasticizers that modulate both the mechanical and acoustic properties of the material. Mechanical properties, including durability, elasticity, hardness, and surface tack and acoustic properties, including attenuation, acoustic impedance, acoustic velocity, and acoustic insertion loss are frequently tuned to meet the requirements of the specific ultrasonic imaging application.

Conventionally, elastomeric acoustic transmission materials have sought to approximate the acoustic properties of water and gel coupling materials, with particular importance placed on matching the velocity of the ultrasonic wave in the material and minimizing its attenuation. However, the requirement to match these acoustic properties has conventionally limited the accessible range of a material's mechanical properties, as the sound velocity and attenuation are functions of a materials elasticity and density. Specifically, the velocity of the ultrasonic wave in a material is a function of its elasticity and density, i.e., c=sqrt(K/rho), where c is the acoustic wave velocity, K is a measure of the material's elasticity (e.g., bulk modulus), and rho is the material's density. Similarly, the attenuation of ultrasonic energy in a material is governed by its acoustic impedance, which is a function of a material's density and acoustic velocity, i.e., Z=rho*c, where Z is acoustic impedance, rho is density, and c is the acoustic velocity.

As a result of these physical relationships, the mechanical and acoustic properties of elastomeric materials are tightly coupled, and adjustment of one parameter often impacts others. Thus, it has been historically difficult for a single class of elastomeric materials to achieve a wide range of mechanical properties while preserving the desired acoustic properties. Consequently, the field has relied on numerous differing material formulations to meet the varying acoustic transmission needs of medical and non-destructive testing applications.

To overcome the limitations of current state of the art approaches for fabricating acoustic transmission materials with a wide range of mechanical properties and narrow range of acoustic properties, the present invention herein describes an approach for producing urethane gel formulations based on, in aspects, a reaction mixture of isocyanate (NCO) prepolymers, selected polyols which contain groups reactive to these isocyanate groups, and organic plasticizers. By modulating the constituents of the reaction mixture according to the current invention, urethanes are produced that vary in stiffness from approximately Shore A50 to less than Shore OOO 0 (i.e., a 10-1000× difference in elastic modulus) while exhibiting a less than 5% variation in the material's acoustic velocity, acoustic impedance, and acoustic attenuation. In acoustic transmission applications, including medical diagnostic imaging, the desired properties of the material are those matched to soft tissue for velocity and impedance, or around 1540 m/s and 1.5 MRayls, respectively. In most instances, the desired attenuation is as low as possible, or at least lower than attenuation of human soft tissue, or less than 1.0 dB/MHz-cm.

Additionally, as described herein, the inventive material's surface properties may also be controlled, from exhibiting no surface tack, to very high surface tack, as may be required by each unique application requiring the use of an acoustic transmission material. The material described herein may also be adhesively bonded to other materials, including silicone rubber, polyurethane rubber, metals, and semi-rigid plastics, thereby providing a means of integrating the material into multi-layer acoustic coupling assemblies, as may be required by each unique application. Various embodiments of the invention are described herein.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

The present invention overcomes limitations of existing elastomeric acoustic transmission materials. The invention, in embodiments, presents an approach for formulating polyurethane-based acoustic transmission materials with a wide range of mechanical properties and narrow range of acoustic properties that are tailored for acoustic transmission in medical, non-destructive testing, sonar, and other acoustic applications.

### BRIEF DESCRIPTION OF THE DRAWINGS

The accompanying drawings illustrate certain aspects of some of the embodiments of the present invention and should not be used to limit or define the invention. Together with the written description, the drawings serve to explain certain principles of the invention. For a fuller understanding of the nature and advantages of the present technology, reference is made to the following detailed description of preferred embodiments and in connection with the accompanying drawings, in which:

FIG. 1 is a schematic of an exemplary acoustic transmission pad used to convey ultrasonic energy into a receiving body in a medical application, according to embodiments of the current invention.

FIG. 2 depicts a multicomponent acoustic transmission apparatus used to convey ultrasonic energy into a receiving body in a medical application in which one of the components is an acoustic coupling agent that facilitates conveyance of ultrasonic energy through the acoustic transmission pad and into a receiving body in a medical application, according to embodiments of the current invention.

FIG. 3 depicts a multicomponent acoustic transmission apparatus used to convey ultrasonic energy into a receiving body in a medical application in which one of the components is an acoustic coupling agent that facilitates conveyance of ultrasonic energy through the acoustic transmission pad and into a receiving body in a medical application and one of the components is an acoustically transmissive protective layer, according to embodiments of the current invention.

FIG. 4 depicts a multicomponent acoustic transmission apparatus used to convey ultrasonic energy into a receiving body in a medical application in which one of the components is an acoustic coupling agent that facilitates conveyance of ultrasonic energy through the acoustic transmission pad and into a receiving body in a medical application and one of the components is an acoustically transmissive protective layer that encapsulates the acoustic transmission pad, according to embodiments of the current invention.

FIG. 5 depicts a multicomponent acoustic transmission apparatus used to convey ultrasonic energy into a receiving body in a non-destructive testing application, according to embodiments of the current invention.

### DETAILED DESCRIPTION OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

The present invention relates to the formulation of polymeric acoustic transmission materials that are based on a reaction mixture of isocyanate (NCO) prepolymers, polyols with OH functionality that are reactive towards NCO groups, and organic plasticizers and fillers. The invention describes a process for the production of these acoustic transmission materials and acoustic coupling apparatuses/articles comprising the acoustic transmission materials.

The physical, acoustic, and mechanical properties of polyurethanes may be modified by altering the composition and reaction conditions of the reaction mixture. Polyurethanes may be tailored to be rigid or flexible, as the polymeric structure consists of soft and hard segments. The soft segments are formed by high-molecular-weight polyols, which influence polyurethane flexibility and elastic properties, while the hard segments are formed by isocyanate and crosslinking agents, which influence rigidity and durability, in general, reduction of the NCO:OH molar ratio in the cured polymer reduces crosslinking within the polymer network and increases the polyurethane's elasticity, flexibility, and softness. However, when forming soft polyurethane gels, the NCO:OH molar ratio must be high enough to form the crosslinked polymer network, while also low enough to result in unreacted polyol chains that yield gel-like relaxation behavior. Depending on the specific formulation, the lower limit for the NCO:OH molar ratio is approximately 0.1, which yields soft, liquid-like gels, with durometers on the Shore OOO scale.

In addition to reducing the NCO:OH ratio, polyurethane physical properties may be modulated by the addition of organic plasticizers. Internal plasticizers are flexible monomers that incorporate directly in a polymer chain, whereas external plasticizers are materials that interact physically with the elastomer, but do not chemically react with the polymer. External plasticizers can provide the greatest latitude in terms of forming specific compound properties, but they are known to migrate and leach out of the material over time. According to the current invention, the use of plasticizers enables the realization of softer, more flexible polyurethanes than could otherwise be achieved at a particular NCO:OH ratio. Polyurethane gels may be comprised of very high ratios of plasticizers, with exceptionally soft gels exceeding 50% plasticizer by mass, in embodiments.

Suitable isocyanates for preparing the prepolymer reaction mixture are preferably aliphatic, cycloaliphatic, or aromatic polyisocyanates with NCO functionality between 2 and 5, but preferably between 2.5 and 4. Aliphatic isocyanates are preferred when optical clarity and weatherability is desired, because they produce polymers with high UV resistance and improved durability and toughness relative to aromatic polyisocyanates. Examples of suitable polyisocyanates include, but are not limited to the following: methylene diphenyl diisocyante (MDI), hexamethylene diisocyanate (HDI), toluene diisocyanate (TDI), isophorone diisocyanate (IPDI) and optionally modified polymeric compositions or combinations of the above, including biurets and trimers.

Suitable polyols for reaction with the isocyanate include those with functionality of 1 to 6 and molecular weights between 1,000 to 20,000. A wide range of polyether or polyester polyols may be utilized to form polyurethanes, but those with functionalities closer to 2, molecular weights between 1,000 and 5,000, a linear polyether backbone structure, and optionally a vinyl content between 5-60% are preferred for realizing soft polyurethane gels. Higher vinyl content imparts more flexibility to the chain and yields better compatibility with other polyols, especially polypropylene glycols (PPG) and polytetramethylene ether glycols (PTMEG). This improved compatibility provides the opportunity to blend polyols and create hybrid prepolymers. Examples of suitable polyols include but are not limited to the following: polyhydroxyl-polyethers, polyhydroxyl-polyesters, polyhydroxyl-polyacetals, polyhydroxyl-polyesteramides, polyhydroxyl-polyamides, polyhydroxyl-polybutadienes, and mixtures thereof.

External plasticizers are preferred for incorporation into the acoustic transmission material because they do not significantly disrupt the integrity of the polymer network. Suitable external plasticizers for incorporation into the acoustic transmission material include those with long chain, linear aliphatic backbones that promote their retention in the hydrophobic polymer network while also imparting high flexibility to the polyurethane elastomer. Examples of suitable plasticizers include, but are not limited to, the following: Di-n-hepytl phthalate (DHP), Di-2-ethylhexyl phthalate (DOP), Diisoctyl phthalate (DIOP), Diisononyl phthalate (DINP), Diisodecyl phthalate (DIDP), Diundecyl phthalate (DUP), Diisodecyl glutarate (DIDG), Di-n-butyl sebacate (DBS), Diisodecyl adipate (DIDA), Dibutoxyethyl adipate (DBEA), Dibutoxyethoxyethyl sebacate (DBEES), tri octyl trimellitate (TOTM), Dioctyl terephthalate (DOTP). Optionally, phenyl, biphenyl, terphenyl, toluene, xylene, and other alkylbenzenes may be incorporated to provide a high mobility external plasticizer within the elastomer to impart additional flexibility and the ability for the elastomer to self-heal following being torn or damaged.

The NCO:OH molar ratio used to create the acoustic transmission material may range from 0.1 to 4, although it is preferably between 0.5 and 2. Low NCO:OH molar ratios (i.e., <1.0), absent the presence of plasticizers, result in weakly cross-linked polyurethane gels that do not retain their shape and exhibit high surface tack. It is preferred to utilize higher NCO:OH ratios (i.e., >1.0) to yield a sufficiently crosslinked polymer network and leverage the use of internal and/or external plasticizers to achieve the desired physical, mechanical, and acoustic properties.

The polyurethane may be formulated using either a one-shot process or a pre-polymer process. In the one-shot process, all components are added to the reaction mixture simultaneously and are mixed with one another, for example. In a prepolymer process, the entire amount of the isocyanate is first reacted with part of the polyol to form a liquid prepolymer with a reduced free isocyanate content of between 1-15% by weight, which is later reacted with the remaining polyol, plasticizers, and catalysts to yield the elastomer, for example. The benefits of using the prepolymer process include reducing the reaction exotherm and improving processability of the isocyanate to improve mixing during the secondary reaction. In the context of formulating the acoustic coupling material, either of these processes may be used.

The acoustic transmission material may be integrated into an acoustic transmission apparatus, suitable for use in medical, non-destructive testing, sonar, or other acoustic transmission applications. In an embodiment, the acoustic transmission material may be cured in a physical mold and later demolded to impart a desired shape for the end application.

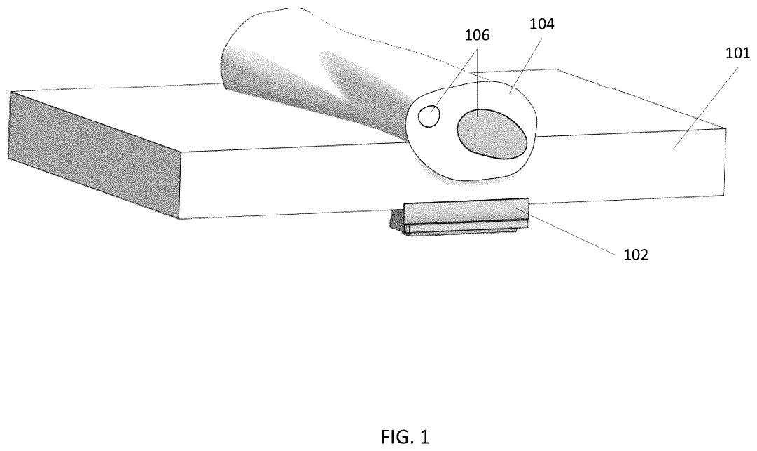

In an embodiment, the polyurethane acoustic transmission material 100 is depicted in FIG. 1 to acoustically couple to an ultrasound transducer 102 and a receiving body 104 having internal features 106 that are the subject of acoustic interrogation. The polyurethane acoustic transmission material 100 can be formulated to have surface tack that provides acoustic coupling between the ultrasound transducer 102, the polyurethane acoustic transmission material 100, and the receiving body 104, which may comprise patient anatomy or a non-destructive testing sample.

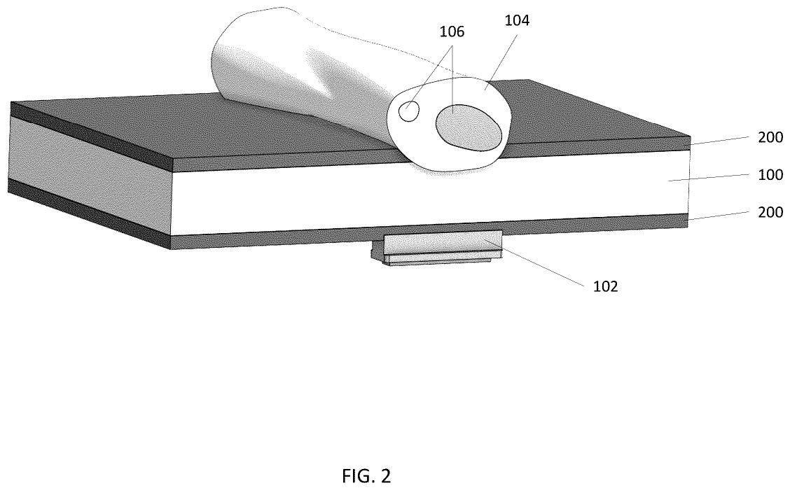

In an embodiment depicted in FIG. 2, an acoustic coupling material 200 is applied to the surface of the polyurethane acoustic transmission material 100 to provide acoustic coupling between the ultrasound transducer 102, the polyurethane acoustic transmission material 100, and the receiving body 104. The acoustic coupling material 200 may comprise an aqueous or non-aqueous mobile phase, preferably comprising water, acoustic coupling gel, hydrogel, aqueous lubricant, synthetic lubricant, mineral oil, or petroleum-based lubricant. The acoustic coupling material 200 may comprise an adhesive that either temporarily affixes or permanently affixes the polyurethane acoustic transmission material 100 to the ultrasound transducer 102 and/or the receiving body 104.

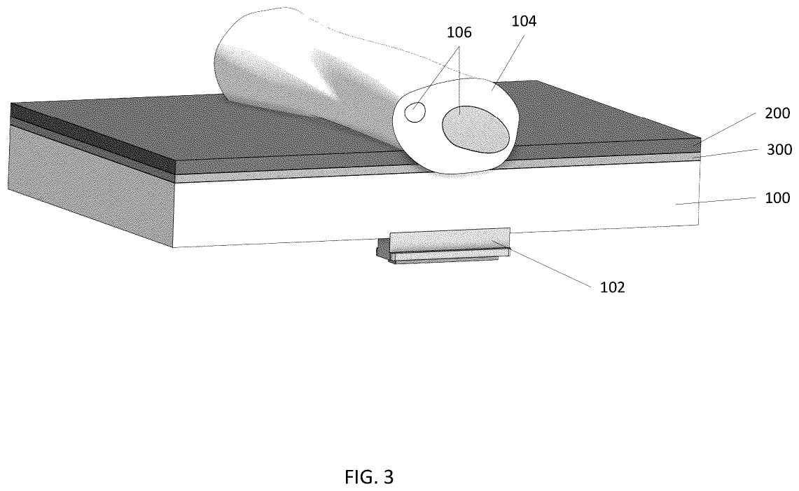

In a preferred embodiment depicted in FIG. 3, the acoustic transmission material 100 can be integrated directly into a multi-component apparatus. An example embodiment is a medical ultrasound apparatus with a biocompatible outer surface material, 300, and an internal space that is filled with the polyurethane-based acoustic transmission material, 100. The biocompatible surface may comprise polyurethane, a cured silicone, or a plastic chosen from one or more of polymethylpentene, cross-linked polystyrene and divinylbenzene, polypropylene, polyether block amide, polyester, polyethylene, polyethylene terephthalate, nylon, and polyimide. The acoustic transmission material 100 can be cured in between the ultrasound transducer 102 and the biocompatible surface 300 thereby providing a means of conveyance of the ultrasonic energy from the ultrasound transducer 102 to the biocompatible surface 300, and ultimately the patient's body 104.

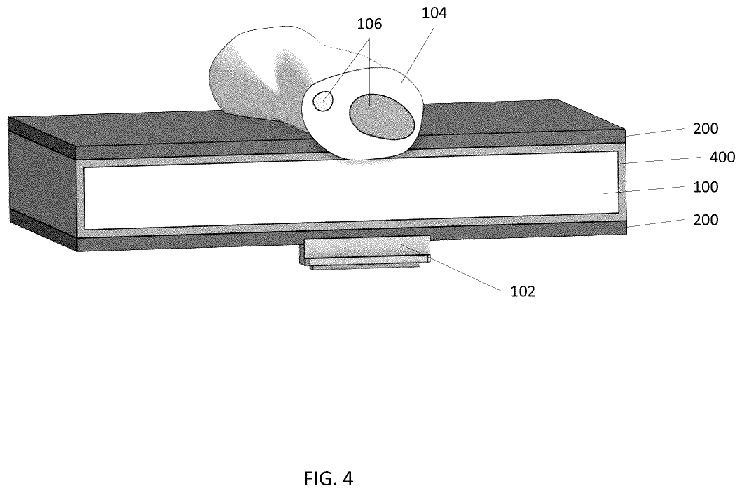

In an embodiment depicted in FIG. 4, the acoustic transmission material 100 can be integrated directly into a multi-component apparatus wherein a biocompatible outer surface material 400 encapsulates the entirety of the polyurethane-based acoustic transmission material 100. The biocompatible surface may comprise polyurethane, a cured silicone, or a plastic chosen from one or more of polymethylpentene, cross-linked polystyrene and divinylbenzene, polypropylene, polyether block amide, polyester, polyethylene, polyethylene terephthalate, nylon, and polyimide. An acoustic coupling material 200 can be applied to the surface of the biocompatible outer surface 400 to provide acoustic coupling between the ultrasound transducer 102, the multi-component apparatus comprising the polyurethane acoustic transmission material 100 and biocompatible outer surface 400, and the receiving body 104. The acoustic coupling material 200 may comprise an aqueous or non-aqueous mobile phase, preferably comprising water, acoustic coupling gel, hydrogel, aqueous lubricant, synthetic lubricant, mineral oil, or petroleum-based lubricant. The acoustic coupling material 200 may comprise an adhesive material that either temporarily affixes or permanently affixes the biocompatible outer surface 400 to the ultrasound transducer 102 and/or the receiving body 104.

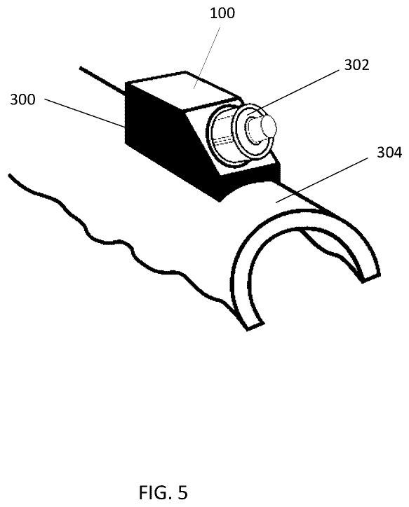

In an embodiment depicted in FIG. 5, a non-destructive testing coupling apparatus has an outer surface coating 300 that can be robust to harsh chemical exposure and an internal space that is filled with the acoustic transmission material 100. In this embodiment, the acoustic transmission material 100 is used as a delay line between a non-destructive testing acoustic transducer 302 and the device undergoing non-destructive testing 304. The acoustic transmission material 100 may be joined to the device undergoing non-destructive testing 304 by either temporarily affixing or permanently affixing the acoustic transmission material 100 to the device undergoing non-destructive testing 304 with an adhesive material or materials. The acoustic transmission material 100 may be joined to the device undergoing non-destructive testing 304 with a mechanical joining mechanism, including by way of example clamps and fasteners. The acoustic transmission material 100 may be joined to the device undergoing non-destructive testing 304 by attaching a mechanical joining mechanism between the outer surface coating 300 and the device undergoing non-destructive testing 304.

In embodiments of these multi-component apparatuses, the polyurethane acoustic transmission material can be cured within a void in the apparatus and not removed. The polyurethane can exhibit adhesion to the surfaces of the other materials in the apparatus, and as is known by those of ordinary skill in the art, adhesion may be improved by use of primers, including by way of example one or more silane-based primer, with properties selected based on the materials involved.

The acoustic transmission material may optionally incorporate additives for conferring additional desired properties. In an example embodiment, the acoustic transmission material formulation may include a trace amount of colorant, like titanium dioxide, for modifying the color of the material. In a separate embodiment, the acoustic transmission material formulation may include microparticle fillers, such as silica microparticles, for modifying the material's acoustic scattering properties.

### Examples

The following isocyanates and blended polyols were used in a one-shot process to produce polyurethane based acoustic transmission materials. Physical, acoustic, and mechanical properties are presented in Table 1.

Polyurethane #1: polymeric hexamethylene diisocyanate trimer with NCO content of 21.8% (Desmodur N 3300, commercially available) reacted with a blended polyol comprised of hydroxyl terminated polybutadiene (Krasol LBH 2000, commercially available from Cray Valley™) and polypropylene glycol (Arcol PPG 2000, commercially available from Covestro™) at an NCO:OH ratio of 1.6. The mixture was formulated to have a hydroxyl functionality of 2, a viscosity of 450 cps, and a density of approximately 0.95 g/ml. Dibutyltin dilaurate (DBTDL) was used as a catalyst, diundecyl phthalate (DUP, Palatinol 111P) was added at 20% w/w, and the mixtures were cured at 80° C. for two hours.

Polyurethane #2: polymeric hexamethylene diisocyanate trimer with NCO content of 21.8% (Desmodur N 3300, commercially available) reacted with a blended polyol comprised of hydroxyl terminated polybutadiene (Krasol LBH 2000, commercially available from Cray Valley™) and polypropylene glycol (Arcol PPG 2000, commercially available from Covestro™) at an NCO:OH ratio of 1.2. The mixture was formulated to have a hydroxyl functionality of 2, a viscosity of 450 cps, and a density of approximately 0.95 g/ml. Dibutyltin dilaurate (DBTDL) was used as a catalyst, diundecyl phthalate (DUP, Palatinol 111P) was added at 20% w/w, and the mixtures were cured at 80° C. for two hours.

Polyurethane #3: polymeric hexamethylene diisocyanate trimer with NCO content of 21.8% (Desmodur N 3300, commercially available) reacted with a blended polyol comprised of hydroxyl terminated polybutadiene (Krasol LBH 2000, commercially available from Cray Valley™) and polypropylene glycol (Arcol PPG 2000, commercially available from Covestro™) at an NCO:OH ratio of 1.2. The mixture was formulated to have a hydroxyl functionality of 2, a viscosity of 450 cps, and a density of approximately 0.95 g/ml. Dibutyltin dilaurate (DBTDL) was used as a catalyst, diundecyl phthalate (DUP, Palatinol 111P, commercially available from BASF™) was added at 20% w/w, terphenyl (Paratherm HT) was added at 5% w/w and the mixtures were cured at 80° C. for two hours.

Polyurethane #4: polymeric hexamethylene diisocyanate trimer with NCO content of 21.8% (Desmodur N 3300, commercially available) reacted with a blended polyol comprised of hydroxyl terminated polybutadiene (Krasol LBH 2000, commercially available from Cray Valley™) and polypropylene glycol (Arcol PPG 2000, commercially available from Covestro™) at an NCO:OH ratio of 1.0. The mixture was formulated to have a hydroxyl functionality of 2, a viscosity of 450 cps, and a density of approximately 0.95 g/ml. Dibutyltin dilaurate (DBTDL) was used as a catalyst, diundecyl phthalate (DUP, Palatinol 111P, commercially available from BASF™) was added at 20% w/w, terphenyl (Paratherm HT) was added at 10% w/w and the mixtures were cured at 80° C. for two hours.

TABLE 1
Physical and Acoustic Properties of Example Acoustic
Transmission Materials Polyurethane #1 through #4.

| | Polyurethane | | | |
|---|---|---|---|---|
| | #1 | #2 | #3 | #4 |
| Formulation | | | | |
| NCO:OH ratio | 1.6 | 1.2 | 1.2 | 1 |
| DUP % w/w | 20 | 20 | 20 | 20 |
| Terphenyl % w/w | 0 | 0 | 5 | 10 |
| Physical Properties | | | | |
| Optical Clarity | Transparent | Transparent | Transparent | Transparent |
| Color | Clear | Clear | Yellow | Yellow |
| Dimensional Stability | Yes | Yes | Yes | Yes |
| Recovery Capacity (sec) | <1 | <1 | 2 | 4 |
| Shore Hardness | Shore A 20 | Shore OO 25 | Shore OOO | Shore OOO |
| | | | 50 | 10 |
| Ability to self heal | No | No | Yes | Yes |
| Surface Tack | None | None | Low | High |
| Acoustic Properties | | | | |
| Speed of Sound (m/s) @ 25 C. | 1510 | 1520 | 1525 | 1530 |
| Attenuation at 5 MHz | −0.71 | −0.74 | −0.71 | −0.67 |
| (dB/MHz/cm) | | | | |
| Acoustic Impedance (MRayl) | 1.5 | 1.5 | 1.5 | 1.5 |

Polyurethane #5: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 12.73% w/w, and the mixtures were cured at 75 C for 1 hour.

Polyurethane #6: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 21.31% w/w, and the mixtures were cured at 75 C for 1 hour.

Polyurethane #7: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 29.41% w/w, and the mixtures were cured at 75 C for 1 hour.

Polyurethane #8: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 36.00% w/w, and the mixtures were cured at 75 C for 1 hour.

Polyurethane #9: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 45.45% w/w, and the mixtures were cured at 75 C for 1 hour.

Polyurethane #10: Hexamethylene diisocyanate oligomers, Isocyanurate with NCO content of 21.89% (Tolonate HDT, commercially available) reacted with a blended polyol comprised of Poly(tetramethylene ether) glycol, 2,2,4-trimethyl-1,3-pentanediol diisobutyrate, Dibutyltin dilaurate, and Anti-foaming Agent (GNX-271RVN13, commercially available from Tandem Products, Inc.™) at an NCO:OH ratio of 1.66. Additional 2,2,4-trimethyl-1,3-pentanediol diisobutyrate was added at a 48.94% w/w, and the mixtures were cured at 75 C for 1 hour.

TABLE 2
Physical and Acoustic Properties of Example Acoustic Transmission
Materials Polyurethane #5 through #7.

| | Polyurethane | | |
|---|---|---|---|
| | #5 | #6 | #7 |
| Formulation | | | |
| NCO:OH ratio | 1.7 | 1.7 | 1.7 |
| Plasticizer % w/w | 12.7 | 12.3 | 29.4 |
| Physical Properties | | | |
| Optical Clarity | Transparent | Transparent | Transparent |
| Color | Clear | Clear | Clear |
| Dimensional Stability | Yes | Yes | Yes |
| Shore Hardness | Shore OOO | Shore OOO | Shore OOO |
| | 45 | 30 | 15 |
| Surface Tack | None | None | None |
| Acoustic Properties | | | |
| Speed of Sound (m/s) @ 25 C. | 1463 | 1437 | 1444 |

TABLE 3
Physical and Acoustic Properties of Example Acoustic Transmission
Materials Polyurethane #8 through #10.

| | Polyurethane | | |
|---|---|---|---|
| | #8 | #9 | #10 |
| Formulation | | | |
| NCO:OH ratio | 1.7 | 1.7 | 1.7 |
| Plasticizer % w/w | 36.0 | 45.5 | 48.9 |
| Physical Properties | | | |
| Optical Clarity | Transparent | Transparent | Transparent |
| Color | Clear | Clear | Clear |
| Dimensional Stability | Yes | Yes | Yes |
| Shore Hardness | Shore OOO | Shore OOO 5 | Shore OOO |
| | 12 | | 5 |
| Surface Tack | Yes | Yes | Yes |
| Acoustic Properties | | | |
| Speed of Sound (m/s) @ 25 C. | 1386 | 1359 | 1326 |

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICWIP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1) An acoustic coupling article for acoustic signal transmission, comprising an elastomeric material capable of conforming to a surface of one or more receiving bodies;
wherein at least one acoustic signal transducer is acoustically coupled to the elastomeric material to transmit acoustic energy produced by the at least one acoustic signal transducer through the elastomeric material into a receiving body;
wherein the elastomeric material comprises a mixture of two or more polymerizable materials, a ratio of the two or more polymerizable materials providing for modulating the Shore Hardness between Shore A50 and Shore OOO 0 and having an acoustic attenuation factor of 3.0 dB/MHz-cm or less.

2) The acoustic coupling article of claim 1, wherein the ratio of the two or more polymerizable materials is capable of being adjusted to give the acoustic coupling a longitudinal speed of sound of between 1000-1850 m/s at 25 C.

3) The acoustic coupling article of claim 1, wherein the ratio of the two or more polymerizable materials is capable of being adjusted to give the elastomeric material an acoustic impedance of between 1.0-3.0 MRayl.

4) The acoustic coupling article of claim 1, wherein the elastomeric material exhibits a density ranging from 0.5 g/cm3-1.5 g/cm3.

5) The acoustic coupling article of claim 1, wherein at least one of the two or more polymerizable materials of the mixture of the two or more polymerizable materials is a polyurethane comprising:
at least one of the following polyols: polyhydroxyl-polyethers, polyhydroxyl-polyesters, polyhydroxyl-polyacetals, polyhydroxyl-polyesteramides, polyhydroxyl-polyamides, polyhydroxyl-polybutadienes, and combinations or mixtures thereof;
at least one of the following isocyanates or composition including one or more isocyanate: methylene diphenyl diisocyanate, hexamethylene diisocyanate, toluene diisocyanate, isophorone diisocyanate, optionally modified polymeric compositions, and combinations or mixtures thereof; and
optionally including additives chosen from one or more of the following: one or more colorant, one or more microparticle filler, and combinations or mixtures thereof.

6) The acoustic coupling article of claim 5, further comprising one or more plasticizers, wherein the one or more plasticizers are added to or combined with the polyurethane to modulate the Shore Hardness, density, acoustic properties, and ability to self-heal following tearing, and wherein the one or more plasticizers include at least one of: Di-n-hepytl phthalate (DHP), Di-2-ethylhexyl phthalate (DOP), Diisoctyl phthalate (DIOP), Diisononyl phthalate (DINP), Diisodecyl phthalate (DIDP), Diundecyl phthalate (DUP), Diisodecyl glutarate (DIDG), Di-n-butyl sebacate (DBS), Diisodecyl adipate (DIDA), Dibutoxyethyl adipate (DBEA), Dibutoxyethoxyethyl sebacate (DBEES), tri octyl trimellitate (TOTM), Dioctyl terephthalate (DOTP), phenyl, biphenyl, terphenyl, toluene, xylene, alkylbenzenes, and combinations and mixtures thereof.

7) The acoustic coupling article of claim 1, wherein the elastomeric material is capable of being molded into a three-dimensional shape, and wherein the molded elastomeric material substantially or mostly retains the three-dimensional shape during use or handling.

8) The acoustic coupling article of claim 1, wherein the elastomeric material is capable of retaining its mechanical and acoustic properties for five years or more.

9) The acoustic coupling article of claim 1, wherein the elastomeric material does not substantially degrade via dehydration or require maintenance to retain its mechanical and acoustic properties for five years or more.

10) The acoustic coupling article of claim 1, wherein the surface of the one or more receiving bodies is a surface or a structural material undergoing non-destructive testing.

11) The acoustic coupling article of claim 1, wherein the surface of the one or more receiving bodies is a surface of an anatomical region of a human body, including at least one of an arm, a leg, a torso, a pelvis, a back, a shoulder, a neck, a head, an abdomen, a chest, a knee, an elbow, a foot, an ankle, a hand, a wrist, a finger, or a toe.

12) The acoustic coupling article of claim 11, wherein the anatomical region of the human body is at least one of an arm, a leg, a torso, a pelvis, a back, a shoulder, a neck, a head, an abdomen, a chest, a knee, an elbow, a foot, an ankle, a hand, a wrist, a finger, or a toe.

13) The acoustic coupling article of claim 1, wherein the elastomeric material is capable of being temporarily affixed to the surface of the one or more receiving bodies, a surface of the at least one acoustic signal transducer, or both the surface of the one or more receiving bodies and the surface of the at least one acoustic signal transducer, using a mechanical joining mechanism or an adhesive material.

14) The acoustic coupling article of claim 1, wherein the elastomeric material is permanently affixed to a surface of the at least one acoustic signal transducer using a mechanical joining mechanism or an adhesive material.

15) The acoustic coupling article of claim 1, wherein the elastomeric material is acoustically coupled to the surface of the one or more receiving bodies, a surface of the at least one acoustic signal transducer, or both the surface of the one or more receiving bodies and the surface of the at least one acoustic signal transducer, using a couplant.

16) The acoustic coupling article of claim 15, wherein the couplant is an aqueous couplant.

17) The acoustic coupling article of claim 16, wherein the aqueous couplant comprises one or more of an acoustic gel, water, saline solution, or a hydrogel.

18) The acoustic coupling article of claim 1, wherein the elastomeric material is acoustically coupled to the surface of the one or more receiving bodies, a surface of the at least one acoustic signal transducer, or both the surface of the one or more receiving bodies and the surface of the at least one acoustic signal transducer, using a non-aqueous couplant.

19) The acoustic coupling article of claim 18, wherein the non-aqueous couplant comprises one or more of a synthetic lubricant, a silicone-based lubricant, a mineral oil, or a petroleum-based lubricant.

20) The acoustic coupling article of claim 1, wherein the elastomeric material is optically transparent or semi-transparent.

21) The acoustic coupling article of claim 1, wherein a surface of the elastomeric material is adhesively bonded to at least one of a silicon rubber, a polyurethane rubber, or a semi-rigid thermoplastic, and wherein the at least one of the silicon rubber, the polyurethane rubber, or the semi-rigid thermoplastic are optionally applied to a silane-based surface primer on the surface of the elastomeric material, thereby forming a multi-layered acoustic coupling article.

22) The acoustic coupling article of claim 21, wherein the multi-layered acoustic coupling article is capable of being temporarily affixed to the surface of the one or more receiving bodies, a surface of the at least one acoustic signal transducer, or both the surface of the one or more receiving bodies and the surface of the at least one acoustic signal transducer, using a mechanical joining mechanism or an adhesive material.

23) The acoustic coupling article of claim 21, wherein the multi-layered acoustic coupling article is permanently affixed to a surface of the at least one acoustic signal transducer using a mechanical joining mechanism or an adhesive material.

24) The acoustic coupling article of claim 21, wherein the multi-layered acoustic coupling article is acoustically coupled to the surface of the one or more receiving bodies, a surface of the at least one acoustic signal transducer, or both the surface of the one or more receiving bodies and the surface of the at least one acoustic signal transducer, using an aqueous couplant.

25) The acoustic coupling article of claim 21, wherein the multi-layered acoustic coupling article is acoustically coupled to the surface of the one or more receiving bodies, a surface of the at least one acoustic signal transducer, or both the surface of the one or more receiving bodies and the surface of the at least one acoustic signal transducer, using a non-aqueous couplant.

26) The acoustic coupling article of claim 21, wherein the surface of the one or more receiving bodies is a surface of an anatomical region of a human body.

27) The acoustic coupling article of claim 21, wherein the surface of the one or more receiving bodies is a surface or a structural material undergoing non-destructive testing.

28) The acoustic coupling article of claim 21, wherein the elastomeric material and any other material it is bonded to are optically transparent or semi-transparent.

29) A composition for acoustically transmitting signals, the composition comprising a selected ratio of polymerizable materials, wherein the selection of isocyanate prepolymers and polyols produces an NCO:OH molar ratio between 0.8 and 2.0, wherein the pre-mixture plasticizer % w/w is between 10% and 60%, wherein the selected ratio provides for modulating a Shore Hardness under Shore A50, and wherein the selected ratio provides for an acoustic attenuation factor of 3.0 dB/MHz-cm or less.

30) An elastomeric material comprising a mixture of two or more polymerizable materials, wherein a ratio of the two or more polymerizable materials is capable of being changed during synthesis of the elastomeric material, the changing of the ratio of the two or more polymerizable materials resulting in a change to a viscosity or a hardness of the elastomeric material, wherein the changing the viscosity or the hardness of the elastomeric material does not result in a five percent or more increase or decrease to the elastomeric material's acoustic velocity, acoustic impedance, and acoustic attenuation.
